(19) United States
(12) Patent Application Publication
(10) Pub. No.: US 2019/0343449 A1
(43) Pub. Date: Nov. 14, 2019

(54) METHOD AND SYSTEM FOR MONITORING CERVIX DILATION TO A DESIRED DIMENSION

(71) Applicant: Ghassan S. Kassab, La Jolla, CA (US)

(72) Inventor: Ghassan S. Kassab, La Jolla, CA (US)

(21) Appl. No.: 16/412,334

(22) Filed: May 14, 2019

Related U.S. Application Data
(60) Provisional application No. 62/671,051, filed on May 14, 2018.

(51) Int. Cl. A61B 5/00; A61B 5/107; A61M 29/02

(52) U.S. Cl. CPC A61B 5/435; A61M 25/10; A61M 29/02; A61B 5/1076

(57) ABSTRACT

A balloon catheter designed for deployment inside a target luminal organ. The balloon catheter having at least one detection element, the detection element configured to collect and send data about the balloon and/or luminal organ to a user operated control device. The control device may then analyze the received data to calculate the status of the luminal organ, such as diameter. The control device may also be configured to notify the user when the luminal organ is in a certain condition.

### PRIORITY

The present U.S. patent application is related to, and claims the priority benefit of, U.S. Provisional Patent Application Ser. No. 62/671,051, filed May 14, 2018, the contents of which are hereby incorporated directly and by reference in their entirety into this disclosure.

### BACKGROUND

At the end of pregnancy, a patient will enter active labor when she is ready to deliver. As part of labor, the cervix will dilate. In the absence of labor, the cervix is typically 5 cm long, and during labor, the cervix can contract to 3.5 cm long as it dilates. The diameter of the cervix must fully dilate to about 10 cm to accommodate the baby's head before the baby can pass through. Once the cervix is fully dilated and the mother begins experiencing contractions, she can push and deliver the baby. This process is called “natural birth.”

During labor, the cervix typically dilates in a predictable manner. Therefore, the dimensions of the cervix are a good indication of how labor is progressing, and the managing healthcare team may obtain the dimensions of the cervix to check on the progression of labor. The current method used by healthcare practitioners to check the dimensions of the cervix is to insert fingers into the vagina. During labor, this procedure is performed many times over to ensure the proper dilatation of the cervix as labor progresses. If the cervix does not dilate over time, a cesarean section procedure may be performed to facilitate childbirth.

There is currently no automatic method to track the progression of the dilatation of the cervix. Repeated insertion of fingers may cause irritation and add to the discomfort of the birthing mother during labor. Further, the continual need to check on a patient is an additional time and resource demand on already busy and stretched medical personnel. Thus, there is a need for a safe, accurate and inexpensive method to track cervical dilatation. This would save time, enhance efficiency and add precision to patient care during labor

### BRIEF SUMMARY

In an exemplary embodiment of a system for monitoring the dilation of a luminal organ of the present disclosure, the device comprises a balloon catheter (which may have an attachment to/with said catheter, or may be wirelessly connected to other componentry, as referenced in further detail herein) comprising an elongate member and a balloon, the balloon disposed on one end of the elongate member and at least one detection element disposed on the balloon catheter; the balloon capable of inflation and deflation and comprising a first diameter, a second diameter and an initial internal pressure; wherein the balloon catheter is capable of being deployed inside a luminal organ, the luminal organ having a dilated diameter and an undilated diameter; wherein the initial internal pressure is sufficient to inflate the balloon to at least the first diameter, the first diameter equal to the undilated diameter such that the balloon contacts the walls of the luminal organ; and wherein the detection element collects and sends data about the balloon or luminal organ to a control device.

The balloon catheter may be positioned inside of the luminal organ and the control device may be positioned exterior to the luminal organ.

In one embodiment the detection element may be a pressure sensor and the initial internal pressure may be sufficient to keep the balloon in contract with the walls of a dilated luminal organ where the balloon is at a second diameter in a dilated luminal organ and the dilated diameter is equal to the second diameter.

The balloon may have an ending internal pressure, and the balloon is at the ending internal pressure when at the second diameter and the control device is configured to send a notification when the pressure sensor detects the balloon is at the ending internal pressure.

In one embodiment, the luminal organ may be the cervix and the second diameter of the balloon may be about 10 cm. The initial internal pressure is about 100 mmHg and the ending internal pressure is about 10 mmHg.

In another embodiment the detection element may be a bio-impedance circuit, and the balloon has a second diameter that is smaller than the dilated diameter.

The balloon may have a slight dumbbell shape.

The data from the detection element is preferably indicative of the dilated or undilated state of the luminal organ, i.e., the extent of dilation. The data may be manipulated and analyzed to determine the dilated or undilated state of the luminal organ.

In one embodiment a balloon catheter is comprised of an elongate member and a balloon having a diameter, the balloon disposed on one end of the elongate member, wherein the balloon is capable of inflation and deflation; wherein the balloon has an internal pressure at a physiological level when disposed in an luminal organ and the diameter is at a first diameter; and wherein the internal pressure of the balloon decreases as the luminal organ opens and the diameter approaches a target diameter.

In a exemplary embodiment of a method for monitoring the dilation of a luminal organ with a balloon catheter, the method comprises the steps of: inserting an uninflated balloon catheter into the luminal organ; where the balloon catheter comprises at least one detection element configured to read and send data about the luminal organ and balloon to an exteriorly positioned control device in communication with the balloon catheter; inflating the balloon to a diameter so that it contacts the walls of the luminal organ; receiving a data about the luminal organ at a control device; and receiving additional data about the luminal organ indicating the luminal organ has dilated at the control device; wherein the additional data indicates a drop in the internal pressure of the balloon or an increase in impedance.

The method may further comprise sending a notification when the additional data indicates the luminal organ has dilated from the control device.

The present disclosure includes disclosure of an exemplary system for monitoring luminal organ dilation, the system comprising a balloon catheter comprising an elongate member and a balloon, the balloon disposed on one end of the elongate member and at least one detection element disposed on the balloon catheter; the balloon capable of inflation and deflation and comprising a first diameter, a second diameter and an initial internal pressure; wherein the balloon catheter is capable of being deployed inside a luminal organ, the luminal organ having a dilated diameter and an undilated diameter; wherein the initial internal pressure is sufficient to inflate the balloon to at least the first diameter, the first diameter equal to the undilated diameter such that the balloon contacts the walls of the luminal organ; and wherein the detection element collects and sends data about the balloon or luminal organ to a control device.

The present disclosure includes disclosure of an exemplary system for monitoring luminal organ dilation, wherein the balloon catheter is positioned inside of the luminal organ and the control device is positioned exterior to the luminal organ.

The present disclosure includes disclosure of an exemplary system for monitoring luminal organ dilation, wherein the detection element is a pressure sensor and the initial internal pressure is sufficient to keep the balloon in contact with the walls of a dilated luminal organ as the luminal organ dilates; and wherein when the balloon is at a second diameter in a dilated luminal organ the dilated diameter is equal to the second diameter.

The present disclosure includes disclosure of an exemplary system for monitoring luminal organ dilation, wherein when the balloon has a ending internal pressure, the balloon being at the ending internal pressure when at the second diameter, and the control device is configured to send a notification when the pressure sensor detects the balloon is at the ending internal pressure

The present disclosure includes disclosure of an exemplary system for monitoring luminal organ dilation, wherein the luminal organ is the cervix and the second diameter is about 10 cm.

The present disclosure includes disclosure of an exemplary system for monitoring luminal organ dilation, wherein the initial internal pressure is about 100 mmHg and the ending internal pressure is about 10 mmHg.

The present disclosure includes disclosure of an exemplary system for monitoring luminal organ dilation, wherein the detection element is a bio-impedance circuit.

The present disclosure includes disclosure of an exemplary system for monitoring luminal organ dilation, the second diameter is smaller than the dilated diameter. The present disclosure includes disclosure of an exemplary system for monitoring luminal organ dilation, wherein the balloon has a dumbbell shape.

The present disclosure includes disclosure of an exemplary system for monitoring luminal organ dilation, wherein the data from the detection element is indicative of the dilated or undilated state of the luminal organ.

The present disclosure includes disclosure of a system for monitoring luminal organ dilation, the system comprising a balloon catheter comprising an elongate member and a balloon having a diameter, the balloon disposed on one end of the elongate member, wherein the balloon is capable of inflation and deflation; wherein the balloon has an internal pressure at a physiological level when disposed in an undilated luminal organ and the diameter is at a first diameter; and wherein the internal pressure of the balloon decreases as the luminal organ dilates and the diameter increases.

The present disclosure includes disclosure of an exemplary system for monitoring luminal organ dilation, wherein the balloon catheter further comprises a detection element disposed on the balloon catheter.

The present disclosure includes disclosure of an exemplary system for monitoring luminal organ dilation, wherein the balloon contacts the undilated luminal organ when at the first diameter.

The present disclosure includes disclosure of an exemplary system for monitoring luminal organ dilation, wherein the detection element is disposed within the balloon and is a pressure sensor.

The present disclosure includes disclosure of an exemplary system for monitoring luminal organ dilation, wherein the detection element is disposed on the balloon and is a bio-impedance circuit.

The present disclosure includes disclosure of an exemplary system for monitoring luminal organ dilation, wherein the balloon comprises a second diameter larger than the first diameter, and the internal pressure of the balloon is lower at the second diameter than at the first diameter.

The present disclosure includes disclosure of a method for monitoring the dilation of a luminal organ with a balloon catheter, the method comprising the steps of inserting an uninflated balloon catheter into the luminal organ, wherein the balloon catheter comprises at least one detection element configured to read and send data to an exteriorly positioned control device in communication with the balloon catheter; inflating the balloon to a diameter so that it contacts the walls of the luminal organ; receiving a data about the luminal organ at a control device; and receiving additional data about the luminal organ indicating the luminal organ has dilated at the control device; wherein the additional data indicates a drop in the internal pressure of the balloon or an increase in impedance.

The present disclosure includes disclosure of an exemplary system for monitoring luminal organ dilation, further comprising sending a notification when the additional data indicating the luminal organ has dilated from the control device.

The present disclosure includes disclosure of an exemplary system for monitoring luminal organ dilation, wherein the balloon is not further inflated as the luminal organ dilates.

The present disclosure includes disclosure of an exemplary system for monitoring luminal organ dilation, wherein the balloon comprises a dumbbell shape and further comprises the step of centering the balloon in the luminal organ.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a lateral cross-section of a balloon catheter, according to a first exemplary embodiment of the present disclosure;

FIG. 2 shows a lateral cross-section of a balloon catheter, according to a second exemplary embodiment of the present disclosure; and

FIG. 3 shows a lateral view cross-section of a balloon catheter, according to a third exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

A method, device, and system to monitor the dilation of a luminal organ are described herein. Although the following description may refer at times to the cervix, it is not intended to limit the use of the present disclosure to the cervix. Rather, it is intended that the present disclosure could potentially be applied to measure the dimensions of any luminal organ or even other pliable openings or organs.

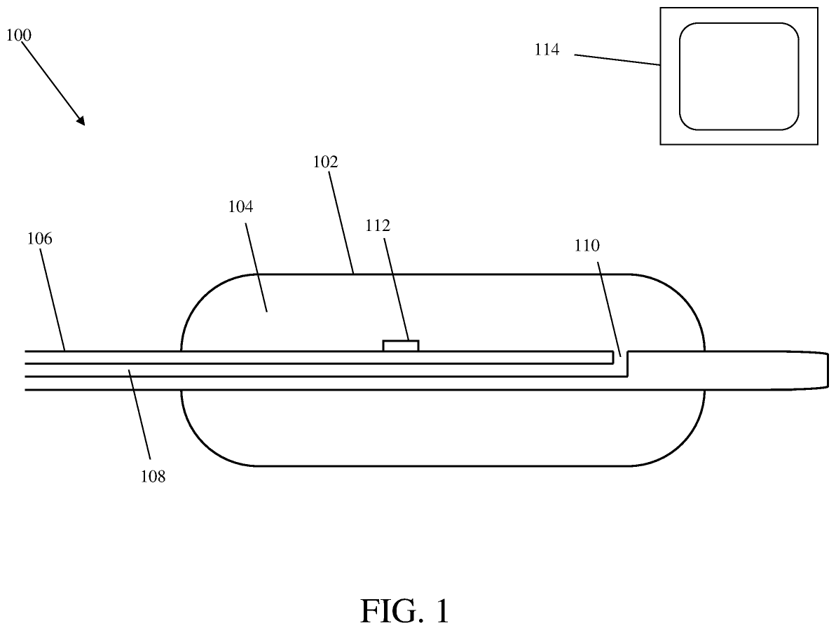

A first embodiment of an exemplary device 100 for measuring the dilation of a luminal organ of the present disclosure is shown in FIG. 1. As shown in FIG. 1, the device comprises a balloon 102 disposed at the end of a catheter 106 to form a balloon catheter. The balloon 102 is constructed so that the expansion and contraction of the balloon 102 and fluid inside will follow the law of gases where PV=nRT, where P, V, n, R, and T correspond to pressure, volume, number of molecules, universal constant and temperature, respectively

In an exemplary device 100 of the first embodiment, the balloon 102 is preferably constructed of a compliant material such as, but not limited to, polyurethane, polyethylene terephthalate, nylon or other standard biomaterial. The balloon 102 may also be constructed to have sufficient excess material so that it may expand through a range of diameters. In an exemplary device for use in the cervix, the balloon 102 is 5 cm long and expandable to larger than 10 cm, 15 cm, 20 cm or greater.

The particular dimensions of the balloon 102 may vary according to the target organ to be measured of the specific anatomy of the patient. For example, in one embodiment designed for monitoring dilation of the cervix during labor, the balloon 102 is about 5 cm long and has a diameter that can expand to at least about 10 cm. Smaller or larger balloons 102 can also be used in accordance with the present disclosure.

The balloon 102 is capable of inflation and deflation, such as by filling or emptying the balloon lumen 104 of a fluid or a gas. The balloon lumen 104 may be filled with air, saline, or other fluid, and may be filled via a lumen 108 and opening in the catheter 106 and or by any other method known in the art. The device further comprises a pressure sensor 112 inside the balloon 102 which may obtain the pressure inside the balloon 102 during the procedure. The pressure information may be sent to a control device 114, as described in more detail below.

During use, the balloon 102 is inflated to an initial known pressure, so that the balloon 102 walls press against the target organ it is inserted into. The volume of the balloon 102 can be calculated from the law of gases, and therefore the dimensions of the balloon 102 may be known. As the balloon 102 is pressing against the walls of the target organ, the balloon 102 and the target organ will share some dimensional measurements, such as diameter. In this embodiment, the balloon will stay in contact with the walls of the luminal organ when fully dilated. As the balloon 102 has been constructed to obey that law of gases, the volume, and therefore the diameter and other dimensions, can be determined from internal pressure measurements.

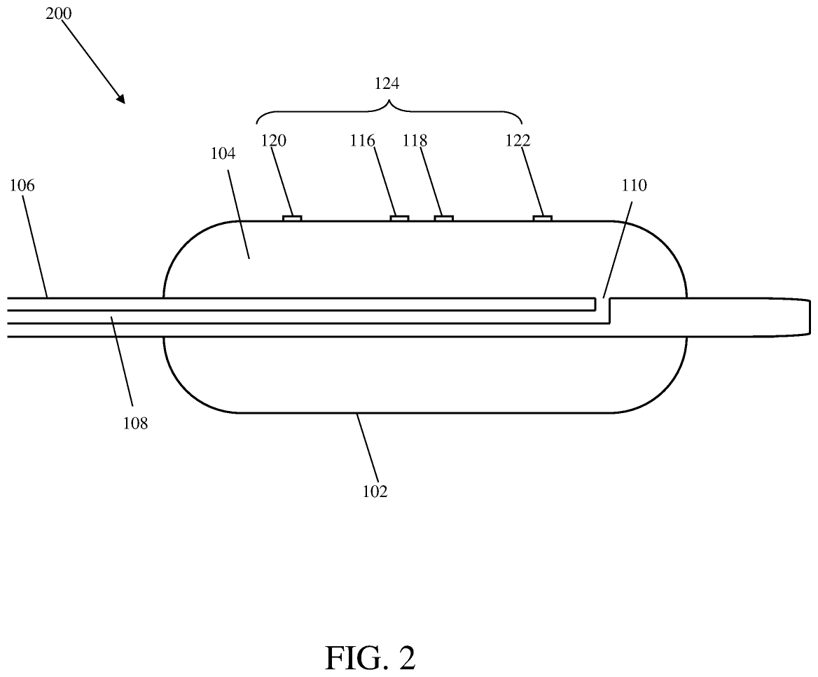

In a second embodiment, such as in FIG. 2, the balloon catheter of the device 200 is constructed generally the same as the first embodiment, but further comprises a bio-impedance electrical circuit 124. The bio-impedance electrical circuit 124 may be in place of, or in addition to, the pressure sensor 112 of the embodiment of FIG. 1. The circuit 124 may be positioned on the outer surface of the balloon 102 such that it makes contact with the luminal organ as the balloon 102 is inflated. In another variation of the second embodiment, the bio-impedance circuit 124 is disposed inside the balloon 102. In various embodiments, exemplary bio-impedance circuits 124 comprise one or more excitation electrodes capable of generating an electrical field and one or more detection electrodes capable of detecting the electrical field. In the second embodiment, the diameter of the target luminal organ is obtained by observing the electrical impedance values.

FIG. 2 illustrates an exemplary device of the second embodiment. The device 200 comprises a balloon 102 disposed on a catheter 106 and is generally constructed similar to the device 100 of FIG. 1. In the device 200 of FIG. 2, the bio-impedance circuit 124 comprises four electrodes disposed on the exterior of the balloon. Two detection electrodes 116, 118 are disposed longitudinally along the balloon 102. A first excitation electrode 120 is disposed longitudinally proximal of the two detection electrodes 116, 118, and a second excitation electrode 122 is disposed longitudinally distal of the two detection electrodes 116, 118. In use, the excitation electrodes 120, 122 generate electrical fields, and the detection electrodes 116, 118 can detect the electrical fields to measure impedance values.

The circuit 124 of FIG. 2 measures bio-impedance during the procedure when the balloon 102 is in contact with the electrically conductive wall of the luminal organ. The balloon is constructed or inflated to a pressure so that the dilated luminal organ will have a larger diameter than the balloon. When the cervix or target luminal organ exceeds the diameter of the balloon 102, the impedance reaches very high values since air is not electrically conductive. As in the device of FIG. 1, the impedance data can be sent to the control device 114 and manipulated and analyzed as needed to diagnose and treat the patient. The impedance information can be used either alone or in conjunction with the pressure measurements.

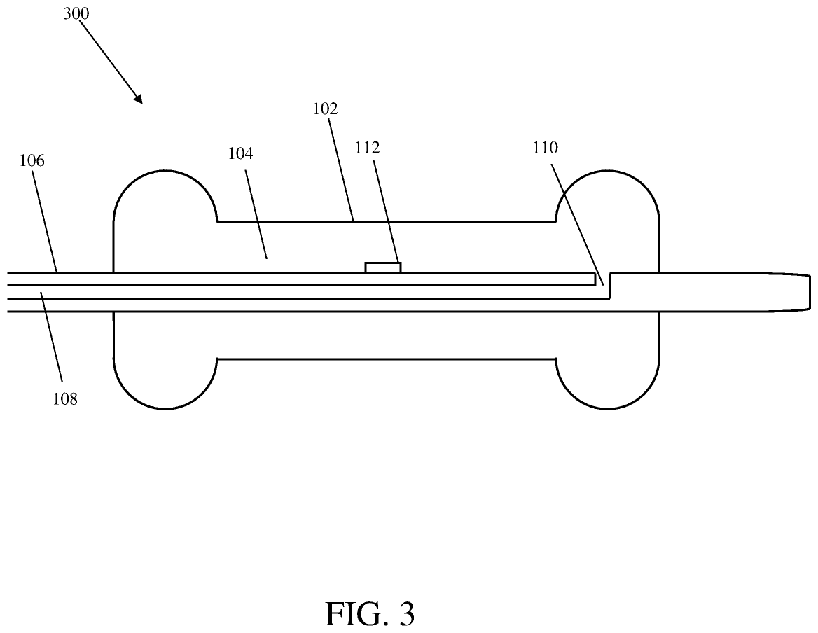

In the third embodiment 300 illustrated in FIG. 3 the balloon catheter is constructed substantially the same as the previously described embodiments. The balloon 102 further comprises a general shape such that it is not easily dislodged from the target organ. As illustrated in FIG. 3, the balloon 102 may have flared or bulbous ends and a relatively narrow waist, such that it has the shape of a dumbbell or dog bone. Such a shape prevents the balloon 102 from unintended movement so that it does not become dislodged externally or penetrates internally.

The balloon 102 may also be fixated onto the women's vagina with adhesive like tape such that it can be held in place during measurements and then easily removed as needed. Tape or other adhesive may be used with any of the previously described embodiments.

The balloon catheter of the previously described embodiments may be in communication with a control device 114 to collect data and send data from the balloon catheter to the control device 114. The balloon catheter may be operably connected to the control device 114 in a wired fashion, wirelessly (such as by radiofrequency (RF), Wi-Fi, Bluetooth®, etc.), or otherwise. The control device 114 may be configured to perform multiple therapeutic and diagnostic functions including, but not limited to, inflating and deflating the balloon, displaying pressure readings, displaying impedance values, calculating and displaying the current dimensions of the luminal organ based on pressure or impedance, and any other related functions. The control device is generally a computer and monitor, but may take different forms, such as small handheld device. Furthermore, there may be multiple control devices 114 used with one balloon. Each device may be capable of the full suite of functions described herein, or devices may only be capable of limited functions, e.g. receiving notifications only.

The control device 114 may be configured to monitor for a condition or multiple conditions based on the balloon 102 status and data returned from the balloon catheter. A non-inclusive list of exemplary conditions include time elapsed, time remaining (such as a countdown timer), balloon pressure, balloon diameter, balloon volume, target organ diameter, rate of organ diameter dilation or constriction, etc. The control device 114 may also be configured to send a notification when one or more conditions are met or not met or there is a combination of met and unmet conditions. The notification may be an audible and/or visual alarm, a text message, phone call or similar. The notification may serve to alert the practitioner or patient that further care is needed. In one embodiment, the user sets the control device to sound an audible alarm when the condition of the balloon pressure reaching a low level like 10 mmHg or less is met.

A method for monitoring the dilation of a luminal organ using an embodiment of the present disclosure is described below. Although the method refers specifically to monitoring the dilation and measuring the dimensions of the cervix, the method can be applied to any embodiment of the present disclosure and any similar measuring procedure.

### Procedure for Measuring and Monitoring Cervix Dilation During Labor

An uninflated balloon is inserted into the cervix through the vagina. The balloon is inflated to a physiological level (around 100 mmHg). It may be necessary to wait a period of time for the cervix to dilate. As the cervix dilates and becomes more compliant, the balloon will increase in volume and the pressure inside the balloon will decrease. The balloon is constructed such that the pressure will decrease gradually to a zero value as the cervix diameter (and therefore the balloon diameter) reaches or exceeds 10 cm. This decrease in pressure follows from the law of gases where PV=nRT. Since nRT is a constant, P decreases with an increase in V.

The pressure sensor collects measurements of the balloon's internal pressure and sends the information to the control device which can display the pressure information for the user. From the pressure information, the control device can calculate the volume of the balloon and therefore the diameter of the balloon and the cervix, which may also be displayed.

The control device may be set to monitor the conditions of a target cervix diameter, a target rate of cervix dilation and/or a target pressure level. If the condition of target cervix diameter (10 cm) is met, if the rate of cervical dilation is not met, or if the pressure level reaches a low level (10 mmHg) an audible alarm will sound alerting the practitioner. The birthing mother is then prepared for pushing, sent to surgery for a cesarean section procedure or evaluated further.

The present disclosure is not intended to be exhaustive or limiting with respect to the content thereof.

While various embodiments of devices and methods for monitoring the diameter and dilation of a luminal organ have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. For example, although the invention has been described in context of measuring the dimension of the cervix, any other luminal organ could also be measured.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A system for monitoring luminal organ dilation, comprising:
a balloon catheter comprising an elongate member and a balloon, the balloon disposed on one end of the elongate member and at least one detection element disposed on the balloon catheter;
the balloon capable of inflation and deflation and comprising a first diameter, a second diameter and an initial internal pressure;
wherein the balloon catheter is capable of being deployed inside a luminal organ, the luminal organ having a dilated diameter and an undilated diameter;
wherein the initial internal pressure is sufficient to inflate the balloon to at least the first diameter, the first diameter equal to the undilated diameter such that the balloon contacts the walls of the luminal organ; and
wherein the detection element collects and sends data about the balloon or luminal organ to a control device.

2. The system of claim 1, wherein the balloon catheter is positioned inside of the luminal organ and the control device is positioned exterior to the luminal organ.

3. The system of claim 1, wherein the detection element is a pressure sensor and the initial internal pressure is sufficient to keep the balloon in contact with the walls of a dilated luminal organ as the luminal organ dilates; and wherein when the balloon is at a second diameter in a dilated luminal organ the dilated diameter is equal to the second diameter.

4. The system of claim 3, wherein when the balloon has a ending internal pressure, the balloon being at the ending internal pressure when at the second diameter, and the control device is configured to send a notification when the pressure sensor detects the balloon is at the ending internal pressure

5. The system of claim 4, wherein the luminal organ is the cervix and the second diameter is about 10 cm.

6. The system of claim 4, wherein the initial internal pressure is about 100 mmHg and the ending internal pressure is about 10 mmHg.

7. The system of claim 1, wherein the detection element is a bio-impedance circuit.

8. The system of claim 7, wherein the second diameter is smaller than the dilated diameter.

9. The system of claim 1, wherein the balloon has a dumbbell shape.

10. The system of claim 1, wherein the data from the detection element is indicative of the dilated or undilated state of the luminal organ.

11. A system for monitoring luminal organ dilation, comprising:
a balloon catheter comprising an elongate member and a balloon having a diameter, the balloon disposed on one end of the elongate member, wherein the balloon is capable of inflation and deflation;
wherein the balloon has an internal pressure at a physiological level when disposed in an undilated luminal organ and the diameter is at a first diameter; and
wherein the internal pressure of the balloon decreases as the luminal organ dilates and the diameter increases.

12. The system of claim 11, wherein the balloon catheter further comprises a detection element disposed on the balloon catheter.

13. The system of claim 11, wherein the balloon contacts the undilated luminal organ when at the first diameter.

14. The system of claim 12, wherein the detection element is disposed within the balloon and is a pressure sensor.

15. The system of claim 12, wherein the detection element is disposed on the balloon and is a bio-impedance circuit.

16. The system of claim 11, wherein the balloon comprises a second diameter larger than the first diameter, and the internal pressure of the balloon is lower at the second diameter than at the first diameter.

17. The present disclosure includes disclosure of a method for monitoring the dilation of a luminal organ with a balloon catheter, the method comprising the steps of:
inserting an uninflated balloon catheter into the luminal organ;
where the balloon catheter comprises at least one detection element configured to read and send data to an exteriorly positioned control device in communication with the balloon catheter;
inflating the balloon to a diameter so that it contacts the walls of the luminal organ;
receiving a data about the luminal organ at a control device; and
receiving additional data about the luminal organ indicating the luminal organ has dilated at the control device;
wherein the additional data indicates a drop in the internal pressure of the balloon or an increase in impedance.

18. The method of claim 17, further comprising:
sending a notification when the additional data indicating the luminal organ has dilated from the control device.

19. The method of claim 17, wherein the balloon is not further inflated as the luminal organ dilates.

20. The method of claim 17, wherein the balloon comprises a dumbbell shape and further comprises the step of centering the balloon in the luminal organ.
